(19) United States
(12) Patent Application Publication
(10) Pub. No.: US 2023/0233190 A1
(43) Pub. Date: Jul. 27, 2023

(54) Actively Cooled Ultrasound Probe with Additively Manufactured Heat Exchanger

(71) Applicant: GE Precision Healthcare LLC, Wauwatosa, WI (US)

(72) Inventors: Reinhold Bruestle, Frankenburg am Hausruck (AT); Caitlin Strobel, New Castle, PA (US); Andrew Desrosiers, West Chester, OH (US); Erich Birglehner, Zeiling (AT); Andreas Kremsl, St. Wolfgang im Salzkammergut (AT); Christian Kreutzer, Puchkirchen am Trattberg (AT)

(21) Appl. No.: 17/581,439

(22) Filed: Jan. 21, 2022

(51) Int. Cl. A61B 8/00; G01S 7/52

(52) U.S. Cl. CPC A61B 8/546; A61B 8/4444; G01S 7/52079

(57) ABSTRACT

An ultrasound probe includes a probe housing, a heat generating electronic component disposed within the housing, and a heat exchanger disposed within the housing and thermally coupled with the heat generating electronic component, wherein the heat exchanger is a monolithic structure without seams. The heat exchanger includes a flow path defined by a plurality of baffles, a fluid inlet connected to one end of the flow path, a fluid outlet connected to the opposite end of the flow path, and one or more turbulating elements disposed within the flow path, the flow path configured for passing a cooling fluid therethrough. The heat exchanger is additively manufactured of a suitable material, such as to form part of a probe central support member.

### BACKGROUND OF THE INVENTION

Embodiments of the present disclosure relate generally to ultrasound imaging probes and, more particularly, to heat dissipating structures of ultrasound imaging probes.

Various medical conditions affect internal organs and bodily structures. Efficient diagnosis and treatment of these conditions typically require a physician to directly observe a patient's internal organs and structures. On many occasions, imaging using an ultrasound imaging system is utilized to obtain images of a patient's internal organs and structures in a minimally invasive manner. The ultrasound images can be obtained utilizing a probe that is located either externally or internally relative to the patient.

By way of example, ultrasound images for non-interventional procedures, such as those obtained for transthoracic echocardiography (TTE), can be obtained by placing the probe against the exterior of the chest of the patient when operating the ultrasound imaging system. Alternatively, ultrasound images for interventional procedures, such as for transesophageal echocardiography (TEE) and/or intracardiac echocardiography (ICE), are obtained by inserting the probe within the body of the patient, e.g., into the esophagus, while the ultrasound imaging system is in operation.

Ultrasound procedures are typically performed in examination, intervention and operating room (open heart surgery) situations where imaging of internal structures of the patient is required. The device utilized in performing the ultrasound procedure typically includes the probe, a processing unit, and a monitor. The probe is connected to the processing unit which in turn is connected to the monitor. In operation, the processing unit sends a triggering signal to the probe. The probe then emits ultrasonic signals via an imaging element within the probe into the patient. The probe then detects echoes of the previously emitted ultrasonic signals. Then, the probe sends the detected signals to the processing unit which converts the signals into images. The images are then displayed on the monitor.

Typically, during the operation of the ultrasound imaging system, the emission of the ultrasonic signals via an imaging element disposed at or near the tip of the probe generates an amount of heat from the imaging element within the probe. In addition, some advanced probes contain application specific integrated circuits (ASICs) with electronics for transmitting and receiving signals from the imaging element. These ASICs also dissipate power and generate heat. Further, the more power utilized by the imaging element and associated ASIC to emit the ultrasonic signals, which enhances the quality of the obtained images, the more heat is generated by the imaging element and ASIC.

In order to dissipate the heat and comply with regulatory requirements limiting the maximum temperature of the probe, prior art probes include various heat dissipation systems. These heat dissipation systems can be formed as a passive system, which rely on heat transmission through various components of the probe to the exterior environment around the probe, or as an active system, which directs a cooling fluid through a heat exchanger disposed within the probe to conduct heat away from the imaging element.

While heat can be conveyed through the plastic housing using a passive system, the amount of heat that can be dissipated on the probe surface is generally limited by the surface temperature and the surface area. Also, the low thermal conductivity of the plastic material forming the housing places significant restrictions on the amount of heat generated by the imaging device that can be dispersed by the passive system. In addition, to enhance the robustness of the probe and to accommodate the required creepage distance for electrical insulation purposes, in many employing a passive heat dissipation system the plastic housing is formed to be relatively thick, increasing the durability of the probe but consequently reducing the thermal conductivity of the housing and therefore inhibiting heat transfer out of the probe via the passive system. As such, the power output of prior art probes employing passive systems, and their corresponding image quality, is necessarily limited by the surface temperature, the surface area and the thermal conductivity of prior art probe structures.

In contrast, active cooling systems have been developed for placement within the probe to increase the amount of heat dissipation capable for the probe beyond the capabilities of the passive dissipation achieved through the housing, thereby significantly improving power output and image quality. As illustrated in FIG. 1, these active cooling systems, such as those disclosed in U.S. Pat. No. 8,475,375, entitled System and Method For Actively Cooling An Ultrasound Probe, the entirety of which is hereby expressly incorporated by reference herein for all purposes, include a probe 100 that includes a heat exchanger 102 positioned in thermal contact with the heat generating electronics 104, e.g., the imaging element(s) and/or ASIC(s), within the probe 100. The heat exchanger 102 includes a fluid inlet 106 and a fluid outlet 108 connected to conduits 110,112 disposed within a cable 114 extending through the cable 114 between the probe 100 and a probe connector 116. adapted to be secured to an ultrasound imaging system (not shown). The connector 116 includes a reservoir 118 including an amount of a cooling fluid 120, which can be a liquid or a gas, that is directed by a pump 122 into a heat exchanger 124. Within the heat exchanger 124 the fluid 120 is contacted by a cooling air flow from fan 126 disposed adjacent the heat exchanger 124. The cooled fluid 120 is pumped out of the heat exchanger 124 and flows along the conduit 110 into the heat exchanger 102 within the probe 100. The cooled fluid 120 is contacted by the heat generated from the electronics 104 which heats the fluid 120 as the fluid flows along the path defined within the heat exchanger 102. The heated fluid 120 subsequently exits the heat exchanger 102 to flow along the conduit 112 back to the fluid reservoir 118 for pumping back to the heat exchanger 124 for cooling by the fan 126. This cycle operates continuously to actively remove the heat from the probe 100 that is generated by the operation of the electronics 104.

In order to enable the fluid 120 to be heated by the heat from the electronics 104 and remove sufficient heat from the probe 100, referring to FIG. 2, the heat exchanger 102 is formed with a tortious internal flow path 128 extending between the fluid inlet 106 and the fluid outlet 108. The path 128 retains the fluid 120 within the heat exchanger 102 for a residence time based upon the flow rate provided by the pump 122 to remove sufficient heat from the electronics 104 to enable continued use of the probe 100.

However, these prior art heat exchangers are formed with a two-piece construction that enables the flow path to be precisely machined into the heat conductive material, i.e., the metal, forming the heat exchanger 102. After machining, the two pieces 130,132 forming the heat exchanger 102 are subsequently secured to one another using suitable fasteners or adhesives to join the pieces 130,132 together to form and seal the heat exchanger 102 and the internal flow path 128. Thus, the heat exchangers 102 formed in this manner are prone to having leaks form between the pieces 130,132. Additionally, the requirement for the machining of the flow path 128 in the pieces 130,132 limits the form of the flow path 128, such as to an elongate channel 134, thereby limiting the effective heat transfer that can be achieved by the heat exchanger 102.

Therefore, it is desirable to develop an improved structure for an ultrasound probe heat exchanger that increases the cooling performance of the probe when in operation. The improved cooling performance of the probe structure enables probes with smaller sizes to be formed that have emission areas similar to prior art probes, as well as allowing increased power to be utilized by the probe for ultrasound signal emission to significantly improve the quality of the resulting images obtained by the probe. The improved cooling performance can also enable the probe to be operated for longer periods of time and/or operated at higher ambient environment temperatures due to the increase in cooling performance.

### BRIEF DESCRIPTION OF THE DISCLOSURE

In one exemplary embodiment of the disclosure, an ultrasound probe includes a probe housing, a heat generating electronic component disposed within the housing, and a heat exchanger disposed within the housing and thermally coupled with the heat generating electronic component, wherein the heat exchanger is a monolithic structure without seams.

According to another exemplary embodiment of the disclosure, a method for forming an ultrasound imaging probe includes the steps of forming a heat exchanger as a monolithic structure without seams and assembling the heat exchanger within a housing for the probe in thermal contact with one or more generating electronic components disposed within the housing.

According to a further exemplary embodiment of the disclosure, an ultrasound imaging system includes a processing unit configured to receive and process acquired ultrasound image data to create ultrasound images derived from the ultrasound image data, a display operably connected to the processing unit to present the created ultrasound images to a user, and an ultrasound imaging probe operably connected to the processing unit to obtain the ultrasound image data, the ultrasound imaging probe having a probe housing, a heat generating electronic component disposed within the housing, and a heat exchanger disposed within the housing and thermally coupled with the heat generating electronic component, wherein the heat exchanger is a monolithic structure without seams.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

In the drawings:

FIG. 1 is a schematic view of a prior art active cooling ultrasound imaging probe.

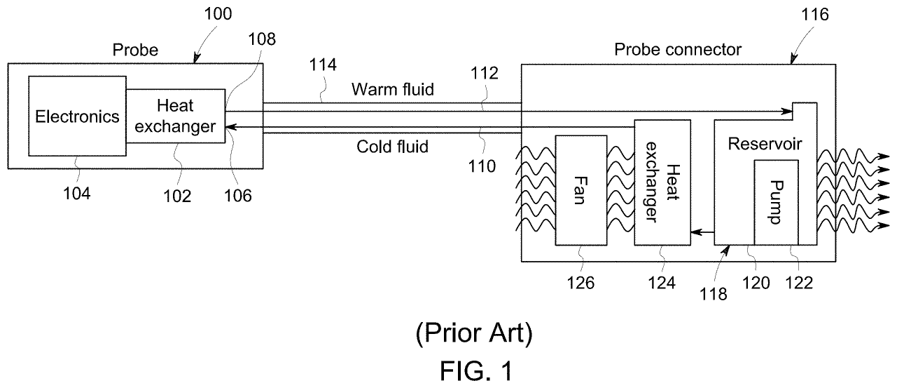

FIG. 2 is an isometric, exploded view of a prior art ultrasound probe heat exchanger.

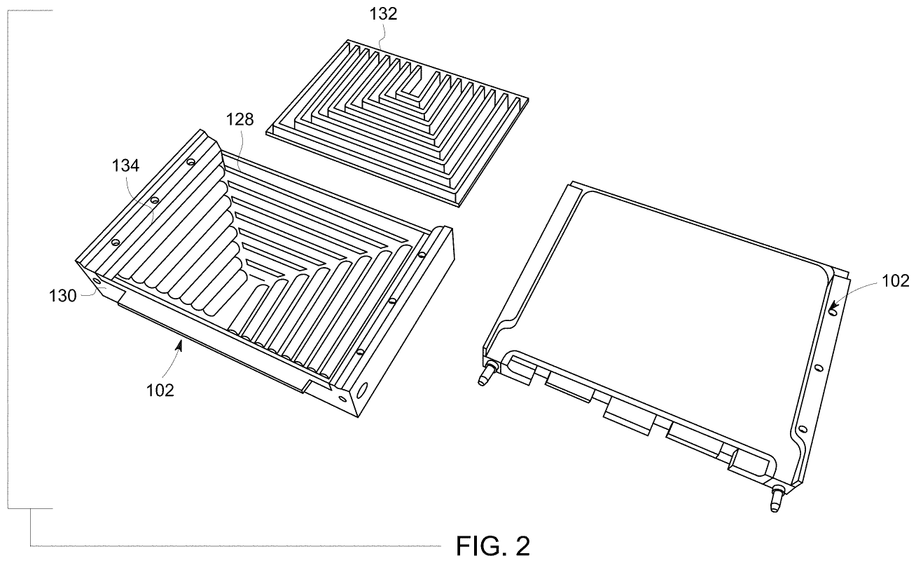

FIG. 3 is a schematic view of an ultrasound imaging system according to an embodiment of the disclosure

FIG. 4 is an isometric view of an ultrasound probe used with the system of FIG. 3 according to an embodiment of the disclosure.

FIG. 5 is a partially broken away, elevational view of a probe connector of the ultrasound probe of FIG. 4.

FIG. 6 is a cross-sectional view along line 6-6 of FIG. 4.

FIG. 7 is a cross-sectional view along line 7-7 of FIG. 4.

FIGS. 8A-8D are cross-sectional views of various embodiments of a heat exchanger disposed within the probe of FIG. 2.

FIGS. 9A-9B are top and side cross-sectional views of another embodiment of the heat exchanger according to an embodiment of the disclosure.

FIGS. 10A-10B are top and side cross-sectional views of another embodiment of the heat exchanger according to an embodiment of the disclosure.

FIGS. 11A-11B are top and side cross-sectional views of another embodiment of the heat exchanger according to an embodiment of the disclosure.

FIGS. 12A-12B are top and side cross-sectional views of another embodiment of the heat exchanger according to an embodiment of the disclosure.

FIGS. 13A-13B are top and side cross-sectional views of another embodiment of the heat exchanger according to an embodiment of the disclosure.

FIG. 14 is an exploded, isometric view of a second embodiment of an ultrasound probe according to an embodiment of the disclosure.

FIG. 15 is an isometric view of a spine of the ultrasound probe of FIG. 14.

FIG. 16 is a partially broken away, isometric view fo the probe of FIG. 14.

### DETAILED DESCRIPTION

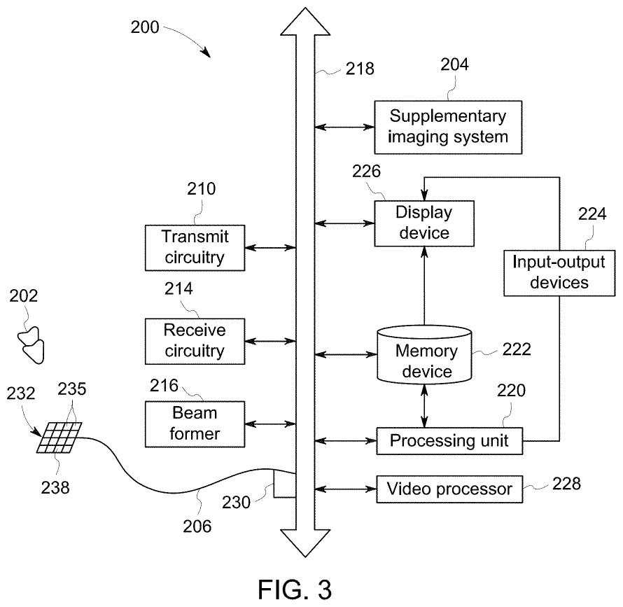

FIG. 3 illustrates an exemplary ultrasound imaging system 200 for optimal visualization of a target structure 202 for use during ultrasound imaging procedures. For discussion purposes, the system 200 is described with reference to an ultrasound probe utilized with the system 200. However, in certain embodiments, other types if imaging probes may be employed with the imaging system 200, such as a TEE probe, a TTE probe, or an ICE probe, among others.

In one embodiment, the ultrasound imaging system 200 employs ultrasound signals to acquire image data corresponding to the target structure 202 in a subject. Moreover, the ultrasound imaging system 200 may combine the acquired image data corresponding to the target structure 202, for example the cardiac region, with supplementary image data. The supplementary image data, for example, may include previously acquired images and/or real-time intra-operative image data generated by a supplementary imaging system 204 such as a CT, MRI, PET, ultrasound, fluoroscopy, electrophysiology, and/or X-ray system. Specifically, a combination of the acquired image data, and/or supplementary image data may allow for generation of a composite image that provides a greater volume of medical information for use in accurate guidance for an interventional procedure and/or for providing more accurate anatomical measurements.

Accordingly, in one embodiment shown in FIG. 3, the ultrasound imaging system 200 includes an interventional device or probe 206 such as an ultrasound probe, a laparoscope, a bronchoscope, a colonoscope, a needle, a catheter and/or an endoscope. The probe 206 is adapted for external use, i.e., the probe 206 is placed on the skin of the patient to image internal structures of the patient, or the probe 206 can be configured to be operated in a confined medical or surgical environment such as a body cavity, orifice, or chamber corresponding to a subject, e.g., the patient.

To that end, in certain embodiments shown in FIG. 3, the ultrasound imaging system 200 includes transmit circuitry 210 that may be configured to generate a pulsed waveform to operate or drive an imaging device 232, which includes one or more transducer elements 236 or a transducer array 238, as controlled by the user via the system 200, or a control device or handle (not shown) operatively connected to the imaging device 232 as part of the system 200. The transducer elements 236 are configured to transmit and/or receive ultrasound energy and may comprise any material that is adapted to convert a signal into acoustic energy and/or convert acoustic energy into a signal. For example, the transducer elements 236 may be a piezoelectric material, such as lead zirconate titanate (PZT), or a capacitive micromachined ultrasound transducer (CMUT) according to exemplary embodiments. The interventional device 206 may include more than one transducer element 236, such as two or more transducer elements 236 optionally arranged in a matrix transducer array 238 or separated from each other on the interventional device 206. The transducer elements 236 produce echoes that return to the transducer elements 236/array 238 and are received by receive circuitry 214 for further processing. The receive circuitry 214 may be operatively coupled to a beamformer 216 that may be configured to process the received echoes and output corresponding radio frequency (RF) signals. The imaging device 132 may be configured to generate cross-sectional images of the target structure 102 for evaluating one or more corresponding characteristics. Particularly, in one embodiment, imaging device 232 is configured to acquire a series of three-dimensional (3D) and/or four-dimensional (4D) ultrasound images corresponding to the subject, though the imaging device 232 can also obtain one-dimensional (1D) and two-dimensional (2D) ultrasound images. In certain embodiments, the imaging system 200 may be configured to generate the 3D model relative to time, thereby generating a 4D model or image corresponding to the target structure, such as the heart of the patient. The imaging system 200 may use the 3D and/or 4D image data, for example, to visualize a 4D model of the target structure 202 for providing a medical practitioner with real-time guidance for navigating the probe 206 on or within the patient.

Further, the system 200 includes a processing unit 220 communicatively coupled to the beamformer 216, the interventional device/probe 206, and/or the receive circuitry 214, over a wired or wireless communications network 218. The processing unit 220 may be configured to receive and process the acquired image data, for example, the RF signals according to a plurality of selectable ultrasound imaging modes in near real-time and/or offline mode.

Moreover, in one embodiment, the processing unit 220 may be configured to store the acquired volumetric images, the imaging parameters, and/or viewing parameters in a memory device 222. The memory device 222, for example, may include storage devices such as a random access memory, a read only memory, a disc drive, solid-state memory device, and/or a flash memory. Additionally, the processing unit 220 may display the volumetric images and or information derived from the image to a user, such as a cardiologist, for further assessment on a operably connected display 226 for manipulation using one or more connected input-output devices 224 for communicating information and/or receiving commands and inputs from the user, or for processing by a video processor 228 that may be connected and configured to perform one or more functions of the processing unit 220. For example, the video processor 228 may be configured to digitize the received echoes and output a resulting digital video stream on the display device 226.

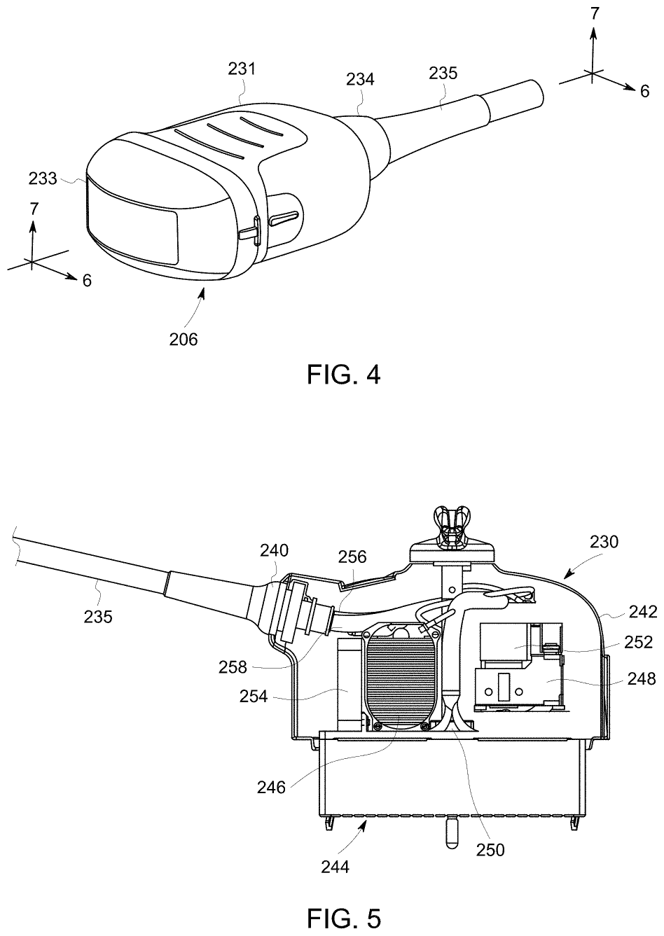

Looking now at the exemplary illustrated embodiment of FIGS. 4-7, the probe 206, is connected to the imaging system 200 using a probe connector 230 and is operable via the system 200 or a control handle (not shown) to control the function and/or movement of the probe 206. The probe 206 includes a handle/housing 231 to which includes a first end 233 that includes the imaging device 232 and a second end 234 that is connected to a cable 235 that extends away from the second end 234 and encloses signal transmission and control/power wiring 237 extending between the system 200 and the probe 206 to control the operation of the imaging device 232.

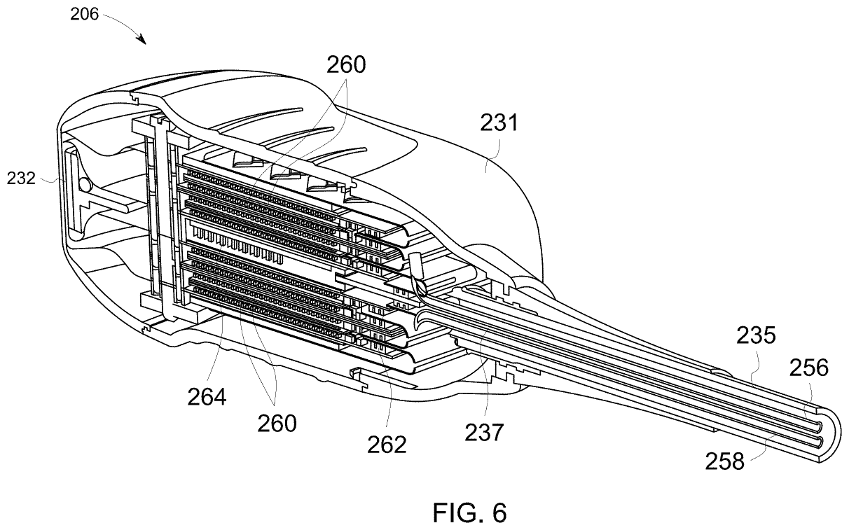

Looking at FIG. 5, opposite the probe housing 231 the cable 235 is engaged with a probe connector 230 that is directly connected to the processing unit 220 to enable the image data obtained from the imaging device 232 to be transmitted to and analyzed by the processing unit 220. The probe connector 230 includes cable connector 240 engaged with the cable 235 and an enclosure 242 having a terminal/plug 244 adapted to be engaged with a complementary receptacle (not shown) located on the processing unit 220.

Within the enclosure 242 are disposed a heat exchanger 246, a fluid reservoir 248, where the fluid can be a liquid or a gas, operably connected to the heat exchanger 246 via a conduit 250, and a pump 252 engaged with the reservoir 248. A fan 254 is also positioned within the enclosure 242 adjacent the heat exchanger 246. In operation, when heated fluid enters the enclosure 242 from the probe 206 via a return tube 256 within the cable 235, the heated fluid is initially directed into the reservoir 248. From the reservoir 248, the heated fluid is moved into the heat exchanger 246 through the conduit 250 by the operation of the pump 252. The heated fluid is directed along the flow path within the heat exchanger 246 while being contacted with a cooling air flow from the adjacent fan 254 to cool the fluid. The cooled fluid is then directed out of the enclosure 242 and back to the probe 206 through a flow tube 258.

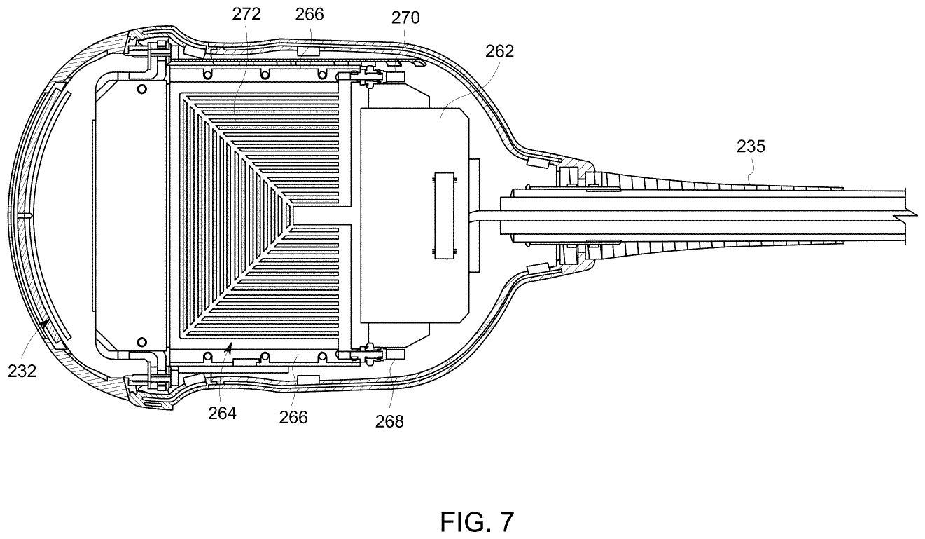

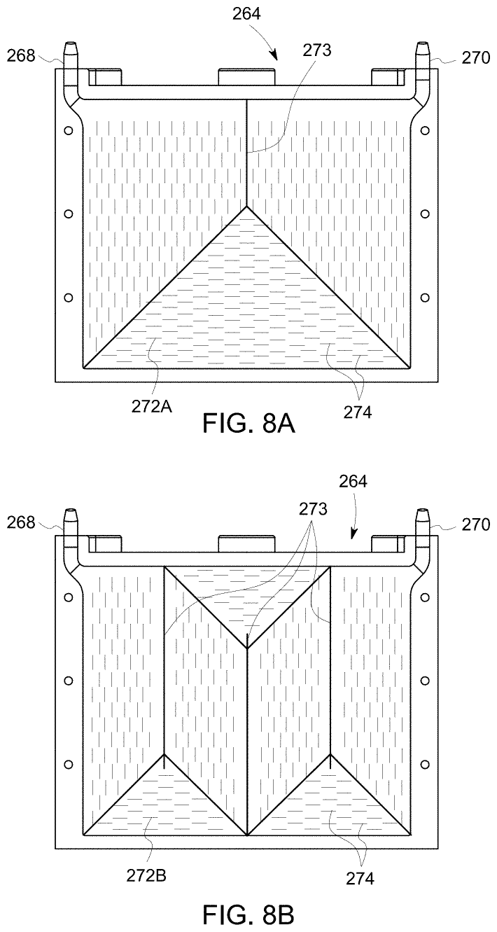

Referring now to the exemplary embodiment of FIGS. 6-7, the housing 231 encloses a number of application specific integrated (ASIC) circuit boards 260 that are utilized to control the operation of the imaging device 232/transducers 236/array 238. The boards 260 are disposed in a stacked configuration with the boards 260 connected to one another and to a control board 262 that is operably connected to the processing unit 220 via the control wiring 237 extending through the cable 235. The ASIC boards 260 are connected to the transducers 236/array 238 opposite the control board 262 in order to send a receive signals from the transducers 236/array 238 while the probe 206 is in operation.

In order to remove the heat generated by the heat generating electronic components in the probe 206, e.g., the imaging device 232/transducers 236/array 238 and the ASIC boards 260 while the probe 206 is operated, one or more heat exchangers 264 are disposed within the stack of ASIC boards 260. The heat exchanger 264 is in direct thermal contact with the heat generating electronic components, e.g., the imaging device 232/transducers 236/array 238 and the ASIC boards 260, and indirectly through the use of one or more side rails 266 engaged with and extending along either side of the stack of ASIC boards 260 and in contact with the heat exchanger 264. Along either or both of the direct or indirect thermal contact or coupling path, heat generated by the transducers 236/array 238 and ASIC boards 260 reaches the heat exchanger 264 for removal from the probe housing 231.

Subsequently, to dissipate heat received by the heat exchanger 264, as best shown in the exemplary embodiment of FIG. 7, the heat exchanger 264 includes a fluid inlet 268 connected to the flow tube 258 and a fluid outlet 270 connected to the return tube 256, which each can include barbs 271 extending outwardly from each of the inlet 268 and outlet 270 for connection to the tubes 256,258 in known manners. The heat exchanger 264 additionally includes one or more flow channels or paths 272 formed within the heat exchanger 264 by walls or baffles 273 formed in the heat exchanger 264 and along which the cooling fluid flows from the fluid inlet 268 to the fluid outlet 270. Similarly to the operation of the heat exchanger 246 in the probe connector 230, the fluid flowing along the flow path in the heat exchanger 264 is contacted by the heat generated by the transducers 236/array 238 and the ASIC boards 260, which is absorbed by the cooling fluid, that is heated as result. The heated fluid subsequently exits the flow path 272 of the heat exchanger 264 and flows along the return tube 258 to the connector 230 to be cooled in the manner described previously prior to being recirculated to the probe 206 for removing additional heat generated by the probe 206.

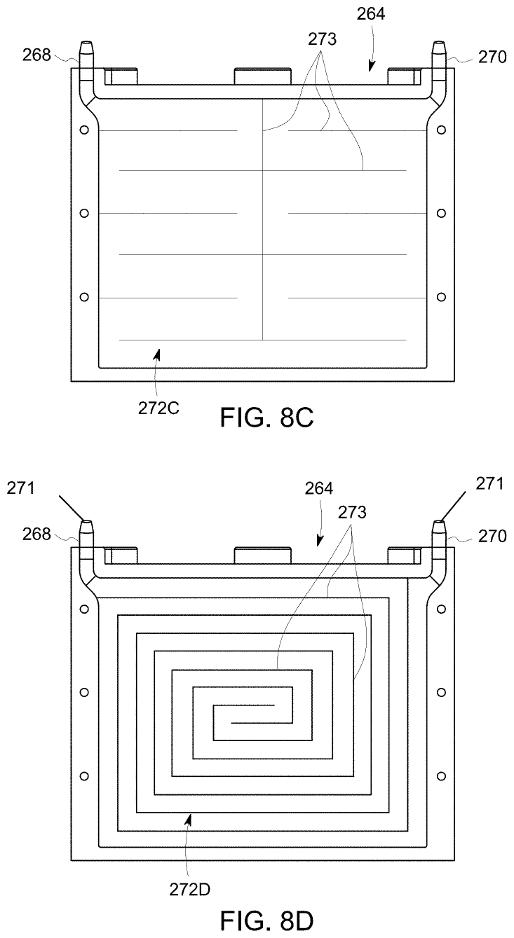

Looking now at FIGS. 8A-13B, with regard to the structure of the heat exchanger 264, the heat exchanger 264 is formed as a monolithic component that defines the flow path 272 therein in an additive manufacturing process. The materials utilized to construct the insert heat exchanger 264 can be selected as desired, and are materials that provide the desired rigidity to the heat exchanger 264, while also enabling heat to be readily transmitted through the heat exchanger 264 material to contact the fluid flowing along the flow path 272 within the heat exchanger 264. In one particular exemplary embodiment, the material forming the heat exchanger 264 is selected from suitable metal materials, including but not limited to aluminum, titanium and copper. In alternative exemplary embodiments, though metals offer improved thermal conductivity, the heat exchanger 264 could also be fabricated from a non-metals, i.e. plastics having the necessary heat conductivity/transfer and structural properties as well as ceramics with high thermal conductivity such as aluminum nitride or boron nitride. These and other materials can be manufactured into the heat exchanger 264 using any suitable additive manufacturing process, including but not limited to vapor chamber printing, as disclosed in U.S. Pat. No. 10,356,945, the entirety of which is hereby expressly incorporated herein by reference for al purposes, powder bed fusion methods including Electron Beam Melting (EBM), Direct Metal Laser Sintering (DMLS), Direct Metal Laser Melting (DMLM), Selective Laser Sintering (SLS), and Binderjet method.

Thus, the heat exchanger 264 is formed without seams between the various surfaces of the heat exchanger 264, negating the needs for bonding or otherwise joining component parts of the heat exchanger 264 to one another and preventing leaks or other failures from occurring within the structure of the heat exchanger 264. Further, the additive manufacturing process enables the heat exchanger 264 to be formed with a more complex geometry for the flow path 272 than is possible with prior art machining manufacturing techniques or processes.

Looking at the exemplary embodiments for the flow path 272 illustrated in FIGS. 7A-7B, heat exchangers 264 are formed with a flow path 272 that has a relatively simple overall geometry, i.e., a U-shaped path 272A with baffles 273 in FIG. 8A and sinuous flow path 272B with baffles 273 in FIG. 8B, but with each flow path 272A,272B including a number of fluid flow turbulating features or elements 274 disposed along the flow path 272A,272B. These elements 274 are spaced from one another, such as in a staggered configuration, to define gaps 276 therebetween, such that a fluid flowing from a fluid inlet 268 to the fluid outlet 270 does not take a linear path through the heat exchanger 264, thereby increasing the heat absorption by the fluid.

In addition, looking at the heat exchangers 264 in FIGS. 8C-8D, the flow paths 272C, 272D defined within these heat exchangers 264 do not include the fluid flow turbulating elements 274, but do form flow paths 272C,272D having baffles 273 with geometries able to be readily formed in the additive manufacturing process for the heat exchangers 264, but not able to be constructed with prior art manufacturing techniques. The increased complexity of the flow paths 272C,272D increases the residence time of the fluid within the flow paths 272C,272D, such that even though the paths allow for generally laminar flow of the fluid along the paths 272C,272D, the fluid can absorb additional heat for removal from the probe 206 as a result.

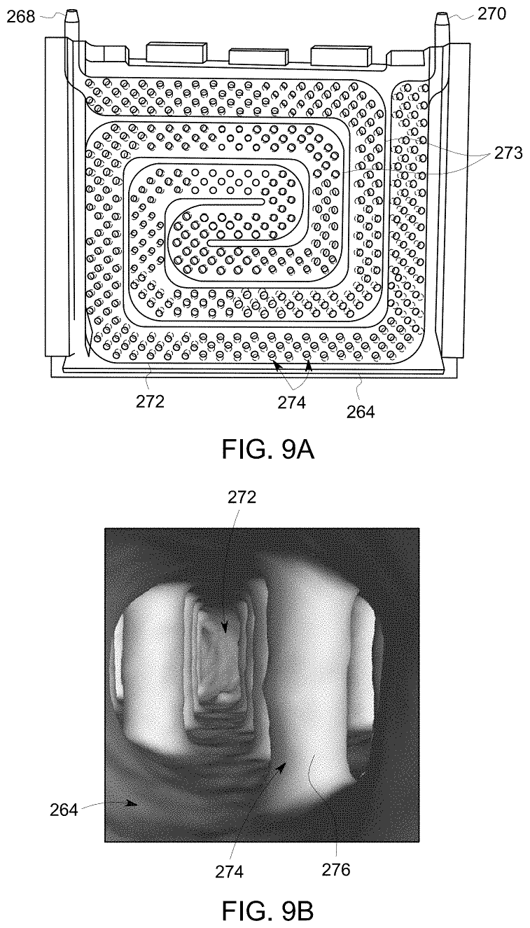

In another particular exemplary embodiment for the heat exchanger 264 shown in FIGS. 9A-9B, the heat exchanger 264 includes spiral flow path 272 defined by baffles 273. The flow path 272 is formed with one or more fluid flow turbulating elements 274 therein, which in the exemplary embodiment of FIGS. 9A-9B are shown in the form of vertical posts 276 extending at least partially across and spaced along the flow path 272. The posts 276 can be formed with any suitable cross-sectional shape and in the illustrated exemplary embodiment are formed with generally circular cross-sections.

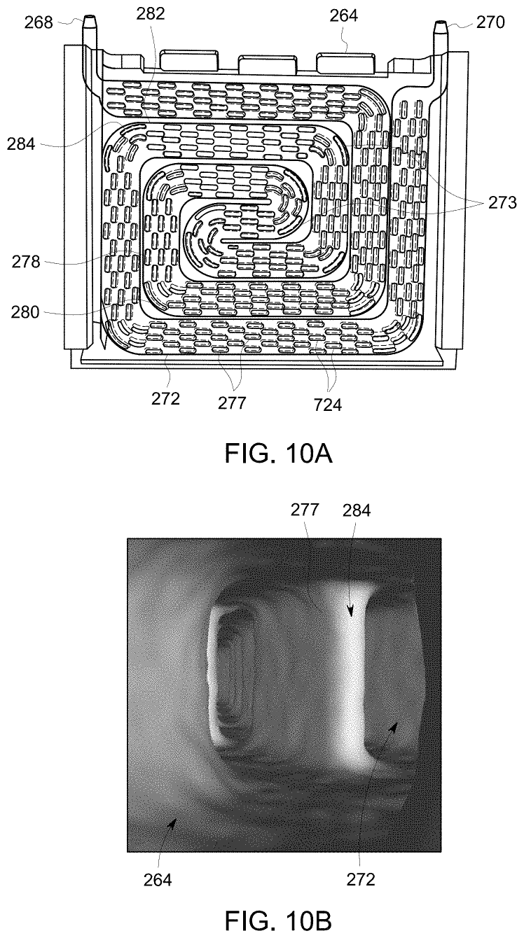

In still another particular exemplary embodiment for the heat exchanger 264 shown in FIGS. 10A-10B, the heat exchanger 264 includes spiral flow path 272 defined by baffles 273. The flow path 272 is formed with one or more fluid flow turbulating elements 274 in the form of vertical walls 277 extending at least partially across and spaced along the flow path 272. The walls 277 in the illustrated exemplary embodiment include walls 277 with flat surfaces 278, curved surfaces 280, and combinations thereof. The walls 277 can also be formed of different lengths depending upon the particular location of the wall 277 within the flow path 272. Further, the leading ends 282 and trailing ends 284 of the walls 277 can be formed with various geometries, i.e., curved, angular, flat, etc., in order to enhance the tubulating/mixing effects of the walls 277 on the fluid flow thought along the flow path 272.

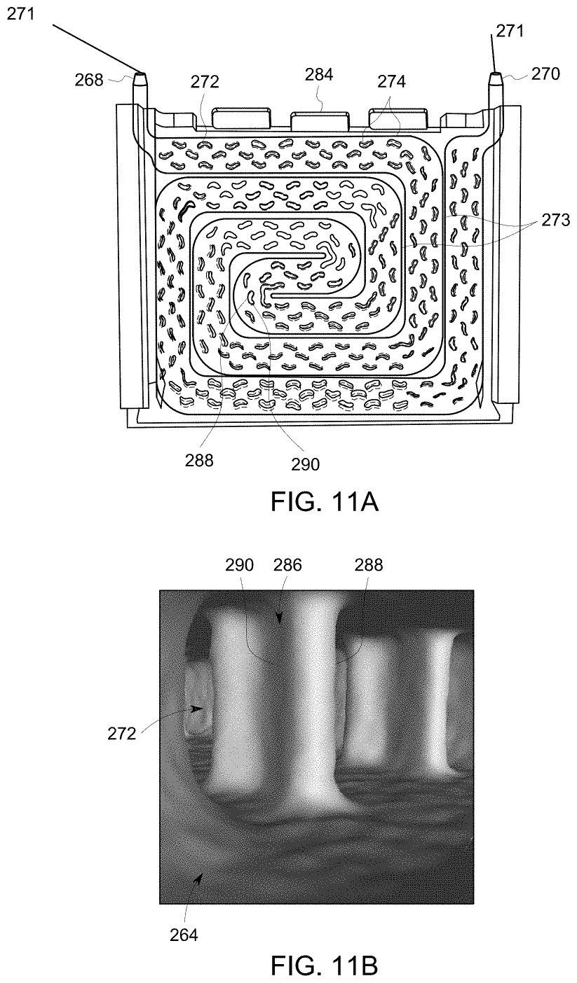

In an exemplary embodiment similar to that of FIGS. 10A-10B, the embodiment of the heat exchanger 264 in FIGS. 11A-11B includes one or more turbulating elements 274 in the form of walls 286 each formed with a convex surface 288 and a concave surface 290 on opposed sides of the wall 286 that extend at least partially across and are spaced along the flow path 272.

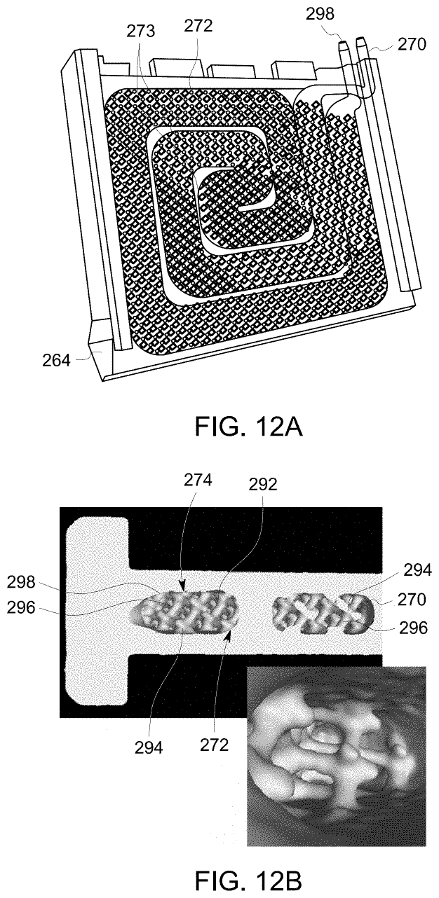

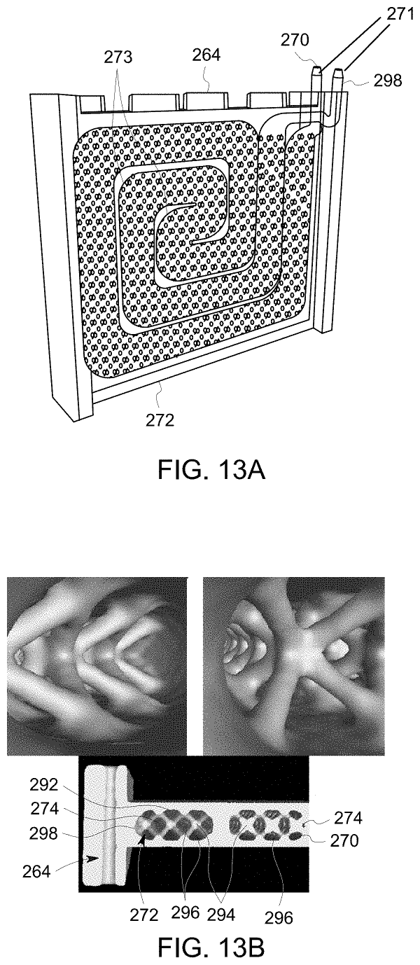

Looking now at the illustrated exemplary embodiments of FIGS. 12A-12B and 13A-13B, the flow path 272 is defined by baffles 273 and is formed with one or more turbulating elements 274 in the form of a lattice 292 extending at least partially across and disposed along the flow path 272. The lattice 292 includes a number of central hubs 294 interconnected with the sides of the flow path 272 and with one another by support columns 296 extending from the hubs 294. The columns 296 can be formed with a perimeter and/or diameter small than that of the hubs 294 to direct the flow of fluid more easily over and around the columns 296 along the flow path 272. In FIGS. 12A-12B, the orientation of the lattice 292 within the flow path 272 is achieved by the formation or additive manufacturing of the heat exchanger 264 at an angle with regard to vertical, such as at an angle of forty-five degrees (45°) from vertical. This provides the lattice 292 with an offset orientation from a lattice 292 constructed in a vertical orientation as illustrated in FIGS. 13A-13B. The ability to form the heat exchanger 264 with the lattice 292 in any orientation for the heat exchanger 264 along the flow path 272 through the use of the additive manufacturing process enables the heat exchanger 264 to provide the increased turbulence to the fluid flowing along the flow path 272 to enhance the heat absorption effects capable using the heat exchanger 264.

Separately from the form of the turbulating elements 274 illustrated in each of FIGS. 12A-13B, both these illustrated exemplary embodiments of the heat exchanger 264 additionally show the use of an impulse canceling fluid inlet 298. The impulse canceling inlet 298 is formed in the heat exchanger 264 closely adjacent and in a parallel direction to the fluid outlet 270. By positioning and orienting the impulse canceling inlet 298 in this manner relative to the fluid outlet 270, and pressure-induced vibrations or other impulses created by the entrance of the fluid into the inlet 298 via a positive displacement pump are reduced and/or canceled out by the pressure-induced vibrations created by the fluid exiting the heat exchanger 264 via the fluid outlet, thereby enhancing the continuous flow of fluid into and out of the heat exchanger 264.

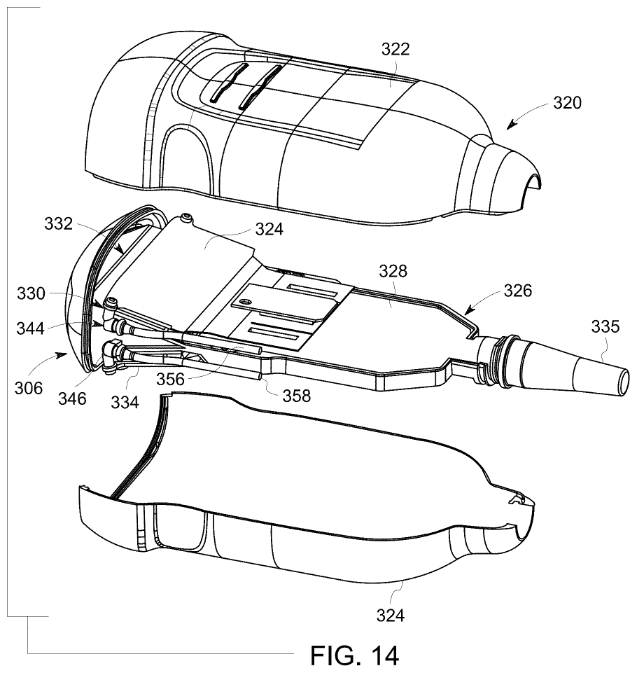

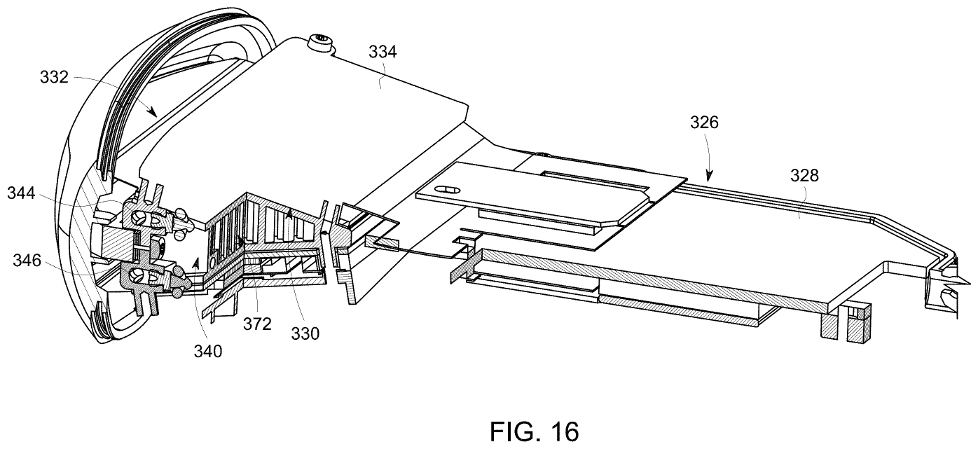

Looking now at FIGS. 14-16, in another exemplary embodiment of the disclosure, the probe 306 is illustrated as including a housing 320 formed of a pair of opposed halves 322,324 joined to one another around a central support member or spine 326. The spine 326 supports a control board 328 that is connected to control and power wiring (not shown) extending through a cable 335 and connected to the ultrasound imaging system 200/processing unit 220. Opposite the wiring, the control board 328 is operably connected to one or more ASIC boards 330 that in turn are operably connected to an imaging device 332 formed with one or more transducer elements/arrays (not shown) which are operated in response to control signals received from the ASIC boards 330 and control board 328. The ASIC boards 330 are secured to the spine 326 and control board 328 by clamps 334 disposed on opposite sides of the spine 326 and secured to the spine 326 over the ASIC boards 330. The clamps 334 operate not only to hold the ASIC boards 330 on the spine 326, but also to direct heat generated by the boards 330 and the imaging device 332 towards the spine 326 along the clamps 334.

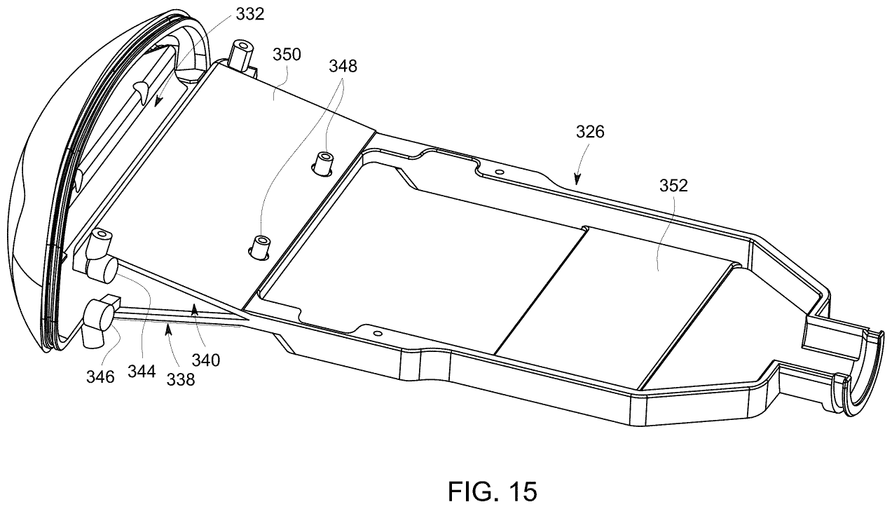

Looking at FIGS. 14-15, a forward end 336 of the spine 326 is formed with a wedge-shape section 338, over which the ASIC boards 330 are positioned. This section 338 of the spine 326 incorporates a heat exchanger 340 formed integrally with the spine 326 and defining a flow path 342 therein. The flow path 342 can have any desired configuration and can have turbulating elements (not shown) similar to those previously described disposed within the flow path 342 to increase the turbulence of the fluid flowing through the heat exchanger 340. The fluid is directed into the heat exchanger 340 though a fluid inlet 344 disposed on one side of the spine 326 and a exits the heat exchanger 340 via a fluid outlet 346 formed on the same side of the spine 326, which are connected to a flow tube 356 and a return tube 358, respectively. In an alternative embodiment, the fluid inlet 344 and the fluid outlet 346 can be formed on opposite sides of the spine 326, such as when using a heat exchanger 340 having a configuration similar to that of FIGS. 12A-13B.

As best shown in FIG. 15, the heat exchanger 340 is formed integrally with the spine 326 in an additive manufacturing process, similar to any of the alternative additive manufacturing methods and processes described previously with regard to other embodiments of the disclosure. In this manner, the heat exchanger 340 can be formed to maximize the space available within the probe 306, thereby enabling the heat exchanger 240 to be formed to provide the maximum amount of heat transfer within the probe 306, as a result of both the overall size and internal configuration for the heat exchanger 340 provided through the use of the additive manufacturing process.

In addition, the heat exchanger 340 can be formed with various external features to facilitate the assembly of the probe 306, such as posts 348 for mounting a thermal transfer pad 350 thereon, where the pad 350 is adapted to support an ASIC board 330 and facilitate the transfer of heat from the board 330 to the heat exchanger 340.

Further, the additive manufacturing process enables the spine 326 to be formed with additional heat transfer components thereon in other locations on the spine 326, such as other heat exchangers (not shown) or a heat sink 352 for the control board 328 to draw additional heat from the probe 306 during operation.

With these enhanced constructions for the heat exchanger 264,340 provided by the additive manufacturing processes and/or methods utilized in the various embodiments, the heat transfer capability of the additively manufactured heat exchangers 264,340 is increased significantly over the prior art machined heat exchangers. In particular, a prior art heat exchanger formed in a conventional machining process has a heat transfer capability of approximately 33 W/m²/K. In contrast, for the embodiment of FIG. 9A the effective heat transfer capability is increased to 135 W/m²/K, an increase of over 4 times that of the prior art machined heat exchanger. Also, the embodiment of FIG. 10A has an effective heat transfer capability of 105 W/m²/K, and the embodiment of FIG. 12A has an effective heat transfer capability of 105 W/m²/K, each a significant increase of the heat transfer capability of the prior art machined heat exchanger.

In alternative embodiments, the heat exchanger 264 can be formed in any of a number of other non-planar configurations, or angled planar configurations, where any turbulating elements 274, if present, can be oriented at an angle with regard to a vertical or horizontal direction. These embodiments for the additively manufactured heat exchanger 264 enable the heat exchanger 264 to be placed in various non-planar locations, e.g., curved or angled, defined within the probe 206,306 and with any perimeter shape in order to maximize the available space within the probe 206,306 for the heat exchanger 264 around the other components located within the probe housing 231,320. In still another alternative exemplary embodiment, the heat exchanger 246 within the enclosure 242 can additionally be formed similarly to heat exchanger 264 as a monolithic structure without seams and with one or more turbulating elements 274.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. (canceled)

2. An ultrasound probe comprising:
a. a probe housing
b. a heat generating electronic component disposed within the housing; and
a heat exchanger disposed within the housing and thermally coupled with the heat generating electronic component, wherein the heat exchanger is a monolithic structure without seams,
wherein the heat exchanger includes a flow path defined by a plurality of baffles, a fluid inlet connected to one end of the flow path and a fluid outlet connected to the opposite end of the flow path, the flow path configured for passing a cooling fluid therethrough.

3. The probe of claim 2, further comprising:
a. a probe cable attached to the probe housing at one end and to a probe connector at the other end, and
b. a pair of tubes extending through the probe cable and attached to the fluid inlet and the fluid outlet, the pair of tubes configured for conveying the cooling fluid from the heat exchanger in the probe housing to and from a second heat exchanger in the probe connector.

4. The probe of claim 3, further including a pump disposed in the probe connector for actively pushing fluid through the heat exchanger in the probe housing, the tubes in the probe cable, and the second heat exchanger in the probe connector.

5. The probe of claim 4, wherein the pump is a positive displacement pump, wherein the fluid inlet is an impulse cancelling fluid inlet disposed adjacent the fluid outlet.

6. The probe of claim 3, wherein the second heat exchanger is a monolithic structure without seams.

7. The probe of claim 2, wherein the flow path includes one or more turbulating elements disposed within the flow path.

8. The probe of claim 7, wherein the one or more turbulating elements comprises a plurality of posts, straight walls, curved walls or lattice structures.

9. The probe of claim 7, wherein the one or more turbulating elements extend across the flow path.

10. The probe of claim 2, wherein the heat exchanger is additively manufactured.

11. The probe of claim 10, wherein the heat exchanger is additively manufactured of a metal.

12. The probe of claim 11, wherein the heat exchanger is additively manufactured of aluminum.

13. The probe of claim 10, wherein the heat exchanger is additively manufactured as part of a probe central support member.

14. (canceled)

15. An ultrasound imaging system comprising:
a. a processing unit configured to receive and process acquired ultrasound image data to create ultrasound images derived from the ultrasound image data;
b. a display operably connected to the processing unit to present the created ultrasound images to a user; and
c. an ultrasound imaging probe operably connected to the processing unit to obtain the ultrasound image data, the ultrasound imaging probe comprising:
i. a probe housing;
ii. a heat generating electronic component disposed within the housing; and
iii. a heat exchanger disposed within the housing and thermally coupled with the heat generating electronic component, wherein the heat exchanger is a monolithic structure without seams, and
wherein the heat exchanger includes a flow path defined by a plurality of baffles, a fluid inlet connected to one end of the flow path, a fluid outlet connected to the opposite end of the flow path, and one or more turbulating elements disposed within the flow path, the flow path configured for passing a cooling fluid therethrough.

16. The ultrasound imaging system of claim 15, wherein the heat exchanger is additively manufactured.

17. The ultrasound imaging system of claim 15, wherein the heat exchanger is additively manufactured as part of a probe central support member.

18. (canceled)

19. A method for forming an ultrasound imaging probe; the method comprising the steps of:
a. forming a heat exchanger as a monolithic structure without seams; and
b. assembling the heat exchanger within a housing for the probe in thermal contact with one or more heat generating electronic components disposed within the housing,
wherein the step of forming the heat exchanger comprises additively manufacturing the heat exchanger to include:
i. a flow path defined by a plurality of baffles,
ii. a fluid inlet connected to one end of the flow path,
iii. a fluid outlet connected to the opposite end of the flow path; and
iv. optionally one or more turbulating elements disposed within the flow path,
wherein the flow path is configured for passing a cooling fluid therethrough.

20. The method of claim 19, wherein the step of forming the heat exchanger comprises additively manufacturing the heat exchanger as part of a probe central support member disposed within the housing.
